(19) United States
(12) Patent Application Publication
(10) Pub. No.: US 2025/0041561 A1
(43) Pub. Date: Feb. 6, 2025

(54) TRANS-EUSTACHIAN MULTI-FUNCTION SYSTEM

(71) Applicant: Alcon Inc., Fribourg (CH)

(72) Inventors: Wesley Hastings Elden, Southlake, TX (US); Venjamin Stojanov, Dietlikon (CH)

(21) Appl. No.: 18/780,993

(22) Filed: Jul. 23, 2024

Related U.S. Application Data
(60) Provisional application No. 63/517,388, filed on Aug. 3, 2023.

(51) Int. Cl. A61M 25/00

(52) U.S. Cl. CPC A61M 25/0026; A61M 2205/0216; A61M 2209/088; A61M 2210/0675

(57) ABSTRACT

The present disclosure generally relates to a trans-eustachian multi-function system. The trans-eustachian multi-function system comprises a body configured for disposition within a eustachian tube, the body comprising a proximal end, a distal end, a transverse cross-sectional shape, a longitudinally-extending length, an outer wall configured for disposition proximate a wall of the eustachian tube, and an inner portion defining a passageway; and a distal anchor disposed proximate the distal end, the distal anchor configured to releasably anchor the body against the wall of the eustachian tube.

### BACKGROUND

Many different conditions affect the middle ear space with resulting hearing loss for the affected patients. Often, the hearing loss needs to be treated by middle ear surgery that requires inserting precision tools into the middle ear space. Diagnosis and surgical treatment, however, present significant challenges due to the difficulty in visualizing the middle ear space, as well as limited accessibility to and the delicate nature of the concerned anatomical structures (e.g., ossicle chain, tympanic membrane, oval window, etc.).

These limitations often make it difficult for surgeons to apply minimally invasive techniques during otological procedures. Instead, they must rely on more invasive surgical approaches (e.g., a postauricular approach or mastoidectomy) to realize sufficiently good visualization of, and accessibility to, the middle ear space to treat a patient's condition successfully.

Accordingly, there is a need in the art for improved devices, systems, and methods for accessing and visualizing the middle ear space to successfully treat conditions that affect the middle ear.

### SUMMARY

Certain embodiments of the present disclosure relate to a trans-eustachian multi-function system. In certain embodiments, a trans-eustachian multi-function system is disclosed, comprising: a body configured for disposition within a eustachian tube, the body comprising a proximal end, a distal end, a transverse cross-sectional shape, a longitudinally-extending length, an outer wall configured for disposition proximate a wall of the eustachian tube, an inner portion defining a passageway; and a distal anchor disposed proximate the distal end, the distal anchor configured to releasably anchor the body against the wall of the eustachian tube.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a schematic cross-sectional view of a human ear.

FIG. 2 illustrates a schematic cross-sectional view of a human eustachian tube and middle ear.

FIG. 3A illustrates a schematic plan view of an embodiment of a trans-eustachian multi-function system, in accordance with certain embodiments.

FIG. 3B illustrates a cross-sectional view taken along Section 3B-3B of an embodiment of a trans-eustachian conduit and passageway of the system of FIG. 3A, in accordance with certain embodiments.

FIG. 3C illustrates a cross-sectional view taken along Section 3C-3C of another embodiment of a trans-eustachian conduit and passageway of the system of FIG. 3A, in accordance with certain embodiments.

FIG. 4A illustrates a schematic plan view of another embodiment of a trans-eustachian multi-function system, in accordance with certain embodiments.

FIG. 4B illustrates a cross-sectional view taken along Section 4B-4B of an embodiment of a trans-eustachian conduit and passageway of the system of FIG. 4A, in accordance with certain embodiments.

FIG. 4C illustrates a cross-sectional view taken along Section 4C-4C of another embodiment a trans-eustachian conduit and passageway of the system of FIG. 4A, in accordance with certain embodiments.

FIG. 5 illustrates a schematic side view of a distal end of a trans-eustachian multi-function system with an embodiment of a distal anchor in an installed state and an uninstalled state, in accordance with certain embodiments.

FIG. 6A illustrates a schematic side view of a distal end of a trans-eustachian multi-function system with another embodiment of a distal anchor in an uninflated and unsealed state within a eustachian tube, in accordance with certain embodiments.

FIG. 6B illustrates a schematic side view of the distal end of the trans-eustachian multi-function system with the distal anchor of FIG. 6A in an inflated and sealed state within a eustachian tube, in accordance with certain embodiments.

FIG. 6C illustrates a schematic side view of a distal end of a trans-eustachian multi-function system with yet another embodiment of a distal anchor in an unexpanded and unsealed state within a eustachian tube, in accordance with certain embodiments.

FIG. 6D illustrates a schematic side view of the distal end of the trans-eustachian multi-function system with the distal anchor of FIG. 6C in an expanded and sealed state within a eustachian tube, in accordance with certain embodiments.

FIGS. 7A-7D illustrate a schematic side view of an embodiment of a trans-eustachian multi-function system comprising a longitudinally extending medical device, in a retracted position (FIG. 7A) and in various stages of insertion (FIGS. 7B-7D), in accordance with certain embodiments.

FIGS. 8A-8C illustrate a schematic side view of another embodiment of a trans-eustachian multi-function system comprising a longitudinally extending medical device, in a retracted position (FIG. 8A) and in various stages of insertion (FIGS. 8B-8C), in accordance with certain embodiments.

FIG. 9 illustrates a schematic side view of an embodiment of a trans-eustachian multi-function system comprising a longitudinally extending medical device comprising an imaging device, in an inserted position, in accordance with certain embodiments.

FIG. 10 illustrates a schematic side view of another embodiment of a trans-eustachian multi-function system comprising a longitudinally extending medical device comprising an imaging device, in an inserted position, in accordance with certain embodiments.

FIG. 11 illustrates a schematic side view of another embodiment of a trans-eustachian multi-function system comprising a longitudinally extending medical device comprising an illumination device, in an inserted position, in accordance with certain embodiments.

FIG. 12 illustrates a schematic side view of another embodiment of a trans-eustachian multi-function system comprising a longitudinally extending medical device comprising an illumination device, in an inserted position, in accordance with certain embodiments.

FIG. 13 illustrates a schematic side view of another embodiment of a trans-eustachian multi-function system comprising a longitudinally extending medical device comprising an aspiration device, in an inserted position, in accordance with certain embodiments.

FIG. 14 illustrates a schematic side view of another embodiment of a trans-eustachian multi-function system comprising a longitudinally extending medical device comprising an infusion device, in an inserted position, in accordance with certain embodiments.

FIG. 15 illustrates a schematic side view of another embodiment of a trans-eustachian multi-function system comprising a longitudinally extending medical device comprising an audio device, in an inserted position, in accordance with certain embodiments.

FIG. 16 illustrates a schematic side view of an embodiment a trans-eustachian multi-function system comprising a plurality of medical device disposed therein, in accordance with certain embodiments.

FIG. 17A illustrates a schematic sectional view of an embodiment of a trans-eustachian multi-function system instrument housing with a plurality of medical devices disposed therein, in accordance with certain embodiments.

FIG. 17B illustrates a cross-sectional front view of the instrument housing of FIG. 17A taken along Section 17B-17B, in accordance with certain embodiments.

FIG. 17C illustrates a perspective front view of the instrument housing of FIG. 17A, in accordance with certain embodiments.

FIG. 18A illustrates a schematic sectional view of another embodiment of a trans-eustachian multi-function system instrument housing with a plurality of medical devices disposed therein, in accordance with certain embodiments.

FIG. 18B illustrates a cross-sectional front view of the instrument housing of FIG. 18A taken along Section 18B-18B, in accordance with certain embodiments.

FIG. 18C illustrates a perspective front view of the instrument housing of FIG. 18A, in accordance with certain embodiments.

FIGS. 19A-19K illustrate various mechanisms for increasing tension of a pull-wire in probes, in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

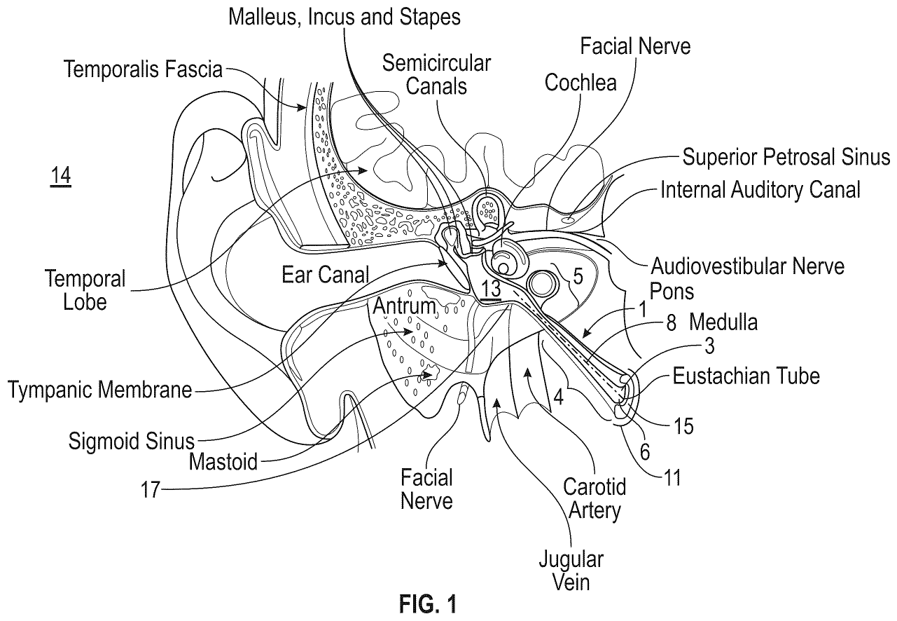

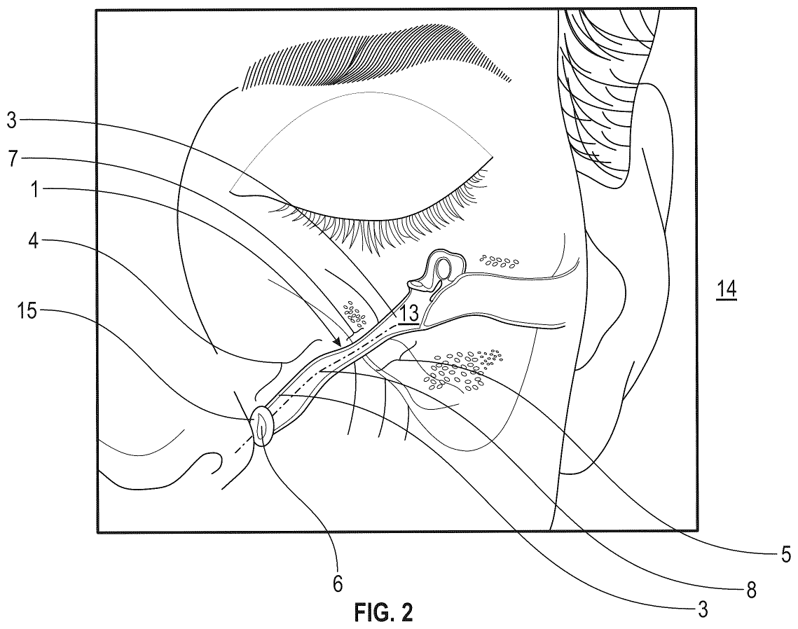

In the following description, trans-eustachian multi-function devices and systems are disclosed. As illustrated in FIGS. 1 and 2, in humans, the eustachian tube 1 is a passageway or tube with an opening at a first end 15 in the nasopharynx 11 that extends to an opening at a second end 17 leading into the middle ear space 13. In certain embodiments, the devices and systems described herein are configured, including in size and shape, for use in humans, and in other embodiments, the devices and systems described herein are configured, including in size and shape, for use in other mammals or animals.

The eustachian tube 1, also known as the auditory tube or pharyngotympanic tube, is a tube with a sidewall or wall 3 that links the nasopharynx 11 to the middle ear space 13, of which it is also a part. In adult humans, the eustachian tube 1 is approximately 35 millimeters (mm) (1.4 in) long and approximately 3 mm (0.12 in) in diameter and follows a pathway substantially defined by a longitudinal axis 8, which may be described as being a series of non-linear line segments, or alternately, as curvilinear. The eustachian tube 1 and longitudinal axis 8 extend from an anterior wall of the middle ear space 13 to a lateral wall of the nasopharynx 11, approximately at the level of the inferior nasal concha. The eustachian tube 1 consists of a bony portion 4 that is about 11-12 mm in length, or approximately ⅓ of the total length of the eustachian tube 1, and a cartilaginous portion 5. The cartilaginous portion 5 of the eustachian tube is about 23-24 mm in length, or approximately ⅔ of the total length, and is formed of a triangular plate of elastic fibrocartilage, the apex of which is attached to the margin of the medial end of the bony portion 4, while its base lies directly under the mucous membrane of the nasal part of the pharynx, where it forms an elevation, referred to as the torus tubarius or cushion, behind the pharyngeal opening of the auditory tube.

The upper edge of the cartilage of the cartilaginous portion 5 is curled upon itself, being bent laterally so as to present on a transverse cross-section the appearance of a hook; a groove or furrow is thus produced, which is open below and laterally, and this part of the eustachian tube 1 is completed by a fibrous membrane. The cartilage lies in a groove between the petrous part of the temporal bone and the great wing of the sphenoid; this groove ends opposite the middle of the medial pterygoid plate. The cartilaginous portion 5 and bony portion 4 of the eustachian tube 1 are not in the same plane, the former inclining downward a little more than the latter. The diameter of the eustachian tube 1 is not uniform throughout, being greatest at a nasopharyngeal opening 6, least at the junction of the bony and cartilaginous portions 4 and 5, respectively, and again increased toward the tympanic cavity; the narrowest part of the eustachian tube 1 is termed the isthmus 7.

The position and relations of the nasopharyngeal opening 6 are described with the nasal part of the pharynx. The mucous membrane of the eustachian tube 1 is continuous in front with that of the nasal part of the pharynx, and behind with that of the tympanic cavity; it is covered with ciliated pseudostratified columnar epithelia and is thin in the osseous or bony portion 4, while in the cartilaginous portion 5 it contains many mucous glands and near the pharyngeal orifice a considerable amount of adenoid tissue.

While the above describes the conventional anatomical perspective of the eustachian tube 1, recent developments in computerized tomography and endoscopic ear surgery have led to further understanding of the eustachian tube 1, including: 1) a view that the bony portion 4 of the eustachian tube is really the anterior extension of the middle ear space 13, or the “protympanum”, and that the term “eustachian tube 1” should or could be limited to the fibrocartilaginous structure connecting the protympanum to the nasopharynx 11; 2) that the eustachian tube 1 may comprise a sac like irregular structure rather than a tubular structure; and 3) that the ear side or end of the eustachian tube 1 is by far the narrowest segment, called the isthmus 7, and is probably the site of possible obstructive pathology causing chronic ear disease.

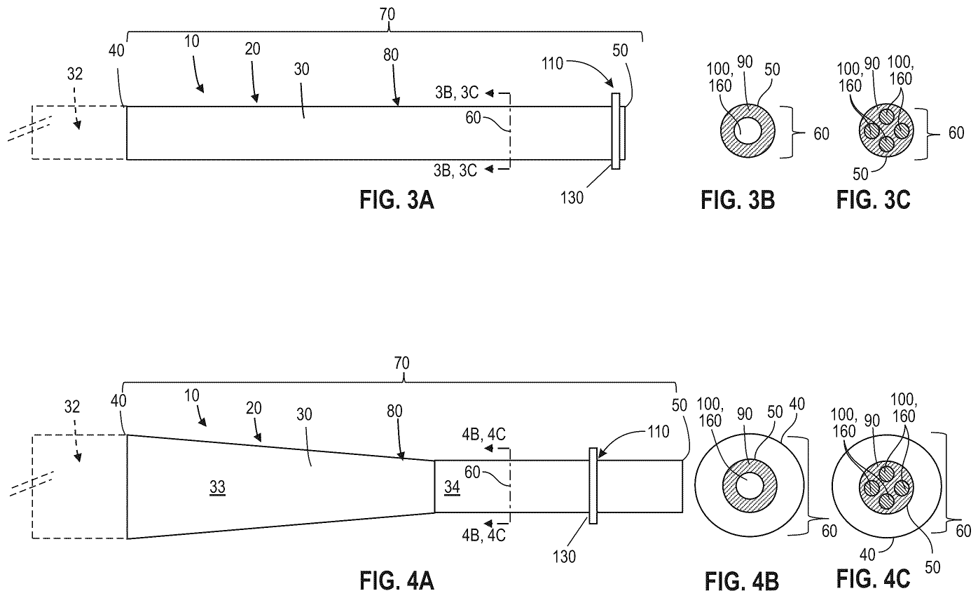

Referring generally now to FIGS. 3-19, in various embodiments, a trans-eustachian multi-function system (hereinafter “system”) 10 is disclosed. The system 10 incorporates multiple elements and features that can be utilized in the eustachian tube 1 to facilitate easier visualization of and/or accessibility to both the eustachian tube 1 and/or middle ear space 13, thereby enabling diagnosis and/or treatment of various eustachian tube and ear conditions and allowing surgeons to reduce invasiveness of surgical procedures, and thus, facilitating better patient outcomes.

The system 10 includes a trans-eustachian probe (hereinafter “probe”) 20 having a trans-eustachian body (hereinafter “body”) 30 configured for disposition within the eustachian tube 1. The body 30 includes a proximal end 40 furthest from the middle ear space 13, a distal end 50 closest to the middle ear space 13, a transverse cross-sectional shape 60, a longitudinally-extending length 70, an outer wall 80 configured for disposition proximate and/or against the wall 3 of the eustachian tube 1, and an inner portion 90 defining at least one trans-eustachian passageway (hereinafter “passageway”) 100. In certain embodiments, the system 10 and probe 20 may also include a distal anchor 110 disposed proximate the distal end 50, the distal anchor configured to releasably anchor the body 30 against the wall 3 of the eustachian tube 1 during use.

The probe 20 generally includes an elongated tubular member that is configured for insertion into the eustachian tube 1 of the patient to provide isolation, including scaling, of the middle ear space 13 from an external (ex vivo) environment, as well as from an internal (in vivo) environment of the patient outside the nasopharyngeal opening 6, including the bodily fluids of the mouth and nasal passages. In this context, isolation may include complete isolation from the external/internal environment, as well as partial isolation, wherein only certain fluids, or controlled amounts of fluids, or the like are permitted to enter or exit around the outer wall 80 of the probe 20. The probe 20 is also configured for insertion into the eustachian tube 1 to provide the at least one passageway 100, namely, an internal passageway for housing, inserting, and/or manipulating various diagnostic, treatment, and/or surgical instruments within the eustachian tube 1, as described herein. The probe 20 and/or body 30 is configured as a sterile, anti-septic member and may be configured for a single sterile use as a disposable member or, alternately, may be configured for cleaning, resterilization, and reuse as a reusable member.

Referring to FIGS. 3A-4C, the body 30 comprises a flexible body and has a size and shape designed in certain embodiments for insertion into, disposition within, and/or general conformance to the shape of the eustachian tube, as described herein. The body 30 is sufficiently flexible to flex or deform and follows the generally non-linear or curvilinear path defined by the longitudinal axis 8 of the eustachian tube 1. The body 30 may have any size and shape suitable for disposition within the eustachian tube 1.

Generally, the body 30 comprises a proximal end 40, a distal end 50, a hollow cylindrical cross-sectional shape 60, and a longitudinally-extending length 70. In certain embodiments, however, the cross-sectional shape 60 is non-cylindrical. In certain embodiments, the body 30 is a discrete element designed for disposition either partially or fully within the eustachian tube 1. In order to support full insertion, the longitudinal length 70 may extend between the proximal end 40 and the distal end 50 and may approximately match the length of the eustachian tube 1. The length 70 may range from 30-40 mm, such as 32-38 mm, and such as 34-36 mm.

Alternately, in other embodiments as shown, for example, in FIGS. 3A and 4A, the body 30 may be part of, or attached to, or configured for attachment to a longer tubular member 32 designed for extending outside the nasopharyngeal opening 6, or even ex vivo, to associated devices and equipment as described herein. In certain embodiments, the tubular member 32 includes, or is attached to, a handle for grasping by a user of the probe 20. In certain embodiments, the tubular member 32 may also be partially disposed within the eustachian tube 1 along with the body 30. In such embodiments, the longitudinal length 70 of the tubular member 32 be any suitable length and extend outwardly beyond the proximal end 40.

In certain embodiments, the entire length 70 of the body 30 is configured to be disposed within the eustachian tube 1. For example, in the embodiments of FIGS. 3A-3C, the body 30 includes an outer wall 80 having a cylindrical shape with a constant or substantially (e.g., +/−10%) constant outer diameter of the outer wall 80 along the length 70. The body 30 of such embodiments may have any suitable outside diameter of the outer wall 80 facilitating insertion and passage of the entire body 30 through the eustachian tube 1, including in certain embodiments a range of 2-6 mm, more particularly 2-5 mm, and even more particularly 2-4 mm. In certain other embodiments, however, the outer wall 80 has a non-cylindrical shape with constant dimensions.

In certain embodiments, a portion of the body 30 is configured to be disposed within the eustachian tube 1, while another portion of the body 30 is configured to remain external to the eustachian tube 1. In other words, only a portion of the body 30 may be sized and shaped for insertion into the eustachian tube 1. For example, in the embodiments of FIGS. 4A-4C, the body 30 with outer wall 80 has a partially tapered/partially straight cylindrical or partially frustoconical/partially cylindrical shape. The body 30 in FIGS. 4A-4C comprises a frustoconical portion 33 having an outer diameter of the outer wall 80 that varies, in some examples continuously or non-continuously, from the larger proximal end 40 along a first portion of the length 70, and a cylindrical portion 34 along a second portion of the length 70 that comprises the same outer diameter along the second portion of the length. The frustoconical portion 33 corresponding to the proximal end 40 has an outside diameter of the outer wall 80 that is greater than the outside diameter of the outer wall 80 at the distal end 50 and the cylindrical portion 34. Thus, the frustoconical portion 33 may be configured to remain external to the eustachian tube 1, while the cylindrical portion 34 may be configured to be disposed in the eustachian tube 1. In certain embodiments, the frustoconical portion 33 may also be configured to seal, or plug, the eustachian tube 1 from the external/internal environments of the patient.

The body 30 of the embodiments of FIGS. 4A-4C may have any suitable outside diameter(s) of the outer wall 80 along the length 70, including those provided herein for the embodiments of FIGS. 3A-3C. In some embodiments, the frustoconical portion 33 of FIGS. 4A-4C may include a taper from a smaller diameter adjacent to the cylindrical portion 34 in a range of 2-6 mm, such as 2-5 mm, and even 2-4 mm, to a larger diameter on the proximal end 40 that does not exceed 8 mm, or 7 mm, or 6 mm, or less.

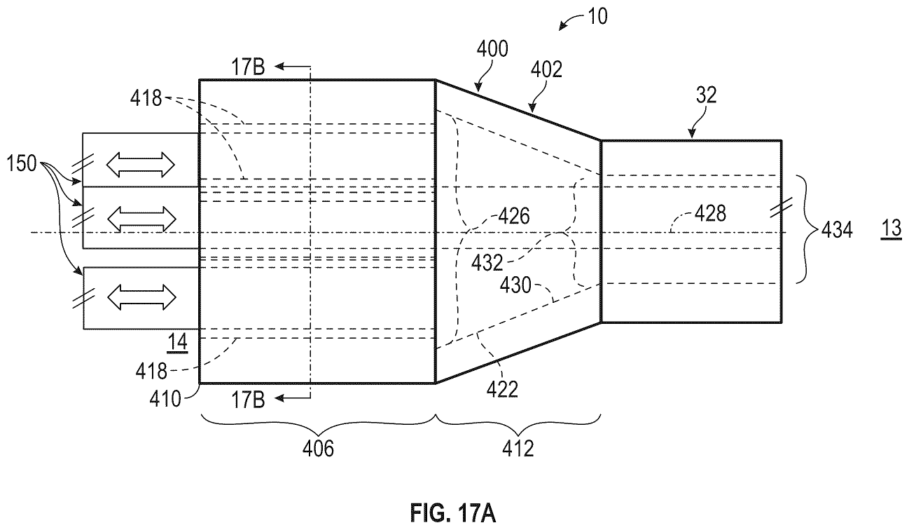

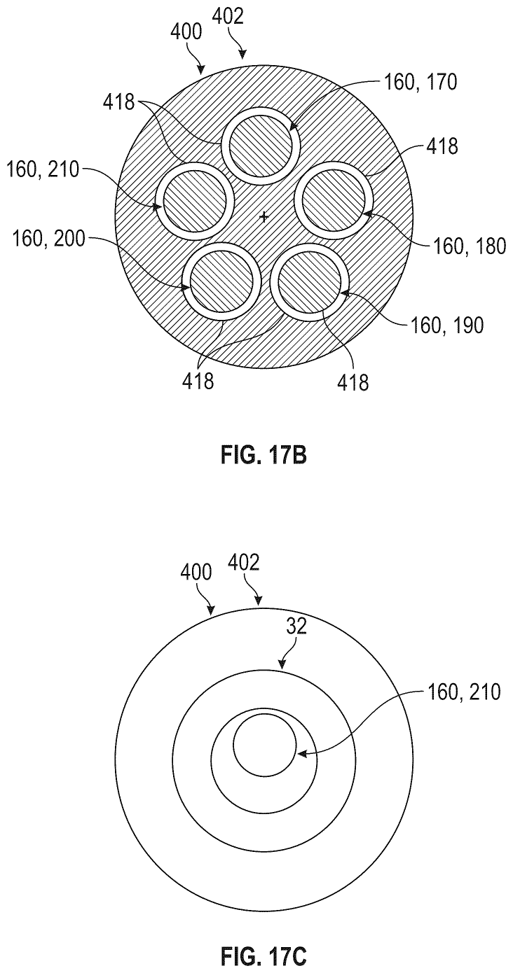

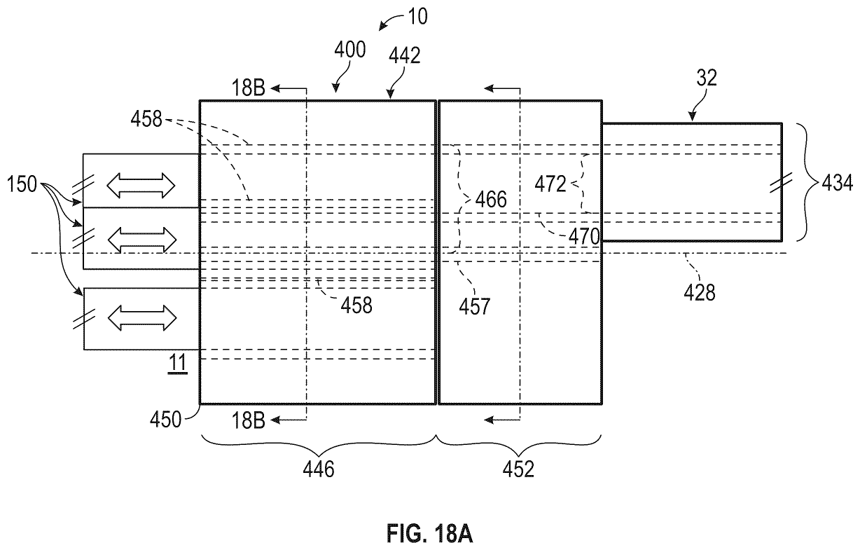

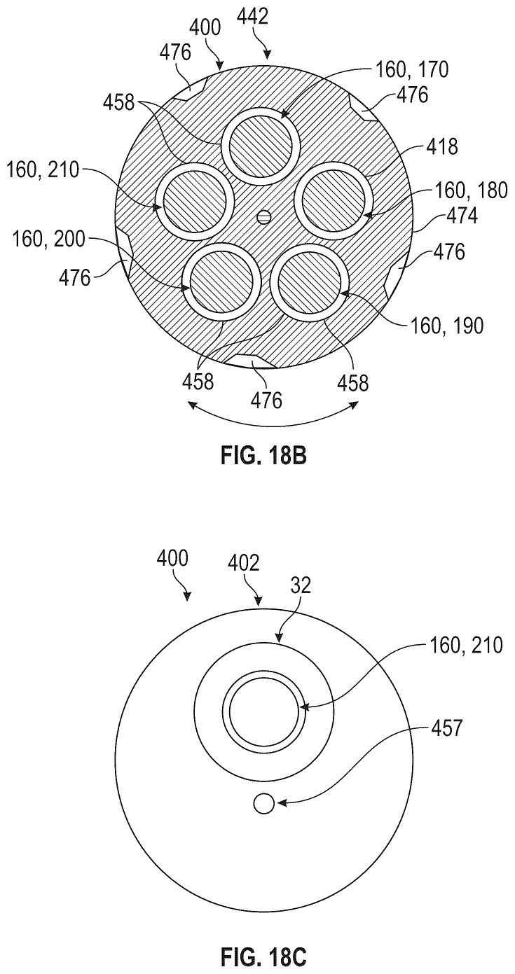

In the embodiments of FIGS. 3B and 4B, the body 30 comprises a single passageway 100 extending internally from the proximal end 40 to the distal end 50 to facilitate passage of one or more medical devices (e.g., instruments) 160 to the distal end 50 for access to the middle ear space 13 when the body 30 is inserted in the eustachian tube 1. The passageway 100 may have any suitable inside dimensions, including in some examples a range of 1-5 mm, more particularly 1-4 mm, and even more particularly 0.5-2.5 mm. The passageway 100 may be cylindrical (or some other non-cylindrical shape with constant lateral dimensions), such as in FIG. 3B, or have a taper in the distal direction, such as in FIG. 4B (e.g., the passageway 100 may have a conical, frustoconical, or other tapered shape). Where the passageway 100 comprises a taper, the taper may facilitate the guidance of one or more of the medical devices 160 as they are urged from the proximal end 40 to the distal end 50 of the body 30 by a user, as described in further detail below with reference to FIGS. 17A-18C.

In the embodiments of FIGS. 3C and 4C, the body 30 comprises a plurality of passageways 100 extending internally from the proximal end 40 to the distal end 50. The plurality of passageways 100 may be disposed within the cross-sectional shape 60 of the body 30 in any suitable manner, including as a plurality of spaced-apart passageways 100, and may comprise any suitable pattern or arrangement of the spaced-apart passageways 100.

The plurality of passageways 100 may comprise any suitable number of passageways, including a range of 2-10 passageways, more particularly 2-6 passageways, even more particularly 2-4 passageways, and the embodiments shown comprising 4 cylindrical and spaced-apart passageways 100. One or more of the passageways 100 may have any suitable lateral or cross-sectional shape, including circular, ellipsoid, and various polygonal shapes that enable ready manipulation of instruments disposed in them as discussed below by longitudinal translation along and/or lateral rotation within the passageway(s) 100. Each one of the plurality of passageways 100 may have any suitable size (i.e., diameter), including in one embodiment a range of 0.25-2.5 mm, more particularly 0.5-2.0 mm, and even more particularly 0.75-2.0 mm, with the proviso that all of the plurality of spaced-apart passageways 100 must be disposed within the cross-sectional shape 60 of the body 30.

In certain embodiments, tomography may be carried out to establish data that may be used to construct a three-dimensional map of the shape and size, including the length, of the eustachian tube 1 and the wall 3 of an individual patient. The data and map may then be processed to determine a slightly undersized model of a body 30 and input into an additive or 3D printer to provide a custom body 30 that is specific to the individual patient and that includes one or more passageways 100 configured to receive the medical devices 160 needed for a specific diagnostic, treatment, or surgical procedure to be performed on the patient.

Generally, the body 30 may be made from any flexible material that is suitable for sterile anti-septic single use as a disposable member, and/or cleaning and resterilization for reuse as a reusable member, and may include various engineering thermoset and thermoplastic polymers, as well as various elastomeric polymers. The body 30 may also comprise various flexible natural and synthetic rubbers.

In certain embodiments, the system 10 and probe 20 comprise a distal anchor 110 disposed proximate the distal end 50, wherein the distal anchor is configured to releasably anchor the body 30 against the wall 3 of the eustachian tube 1. The distal anchor 110 also provides a seal (i.e., a sealing surface) that provides pressed engagement against the wall 3 of the eustachian tube 1 to provide isolation of the middle ear space 13 from the external (ex vivo) environment outside of (e.g., the nasopharynx 11), as well as from the internal (in vivo) environment (e.g., the middle ear space 13, and/or within the body but outside the eustachian tube 1 and nasopharyngeal opening 6), including the bodily fluids of the mouth and nasal passages.

Any device suitable for anchoring the body 30 against the wall 3 of the eustachian tube may be used as the distal anchor 110. Anchoring may comprise providing sufficient resistance to movement of the body 30 against the wall 3 of the eustachian tube 1 to maintain the position of the body as it is used with the instruments as described herein. In this context, isolation may include complete isolation from an external environment 14, as well as partial isolation, wherein only certain fluids, or controlled amounts of fluids, or the like, are permitted to enter or exit around the distal anchor 110 and along the outer wall 80 of the probe 20 under certain conditions, such as to avoid over pressurization or under pressurization (i.e., over evacuation) of the middle ear space 13, for example.

In various embodiments of the system 10, distal anchor 110 may comprise a selectively inflatable/deflatable member, a compressible member, or a selectively expandable member.

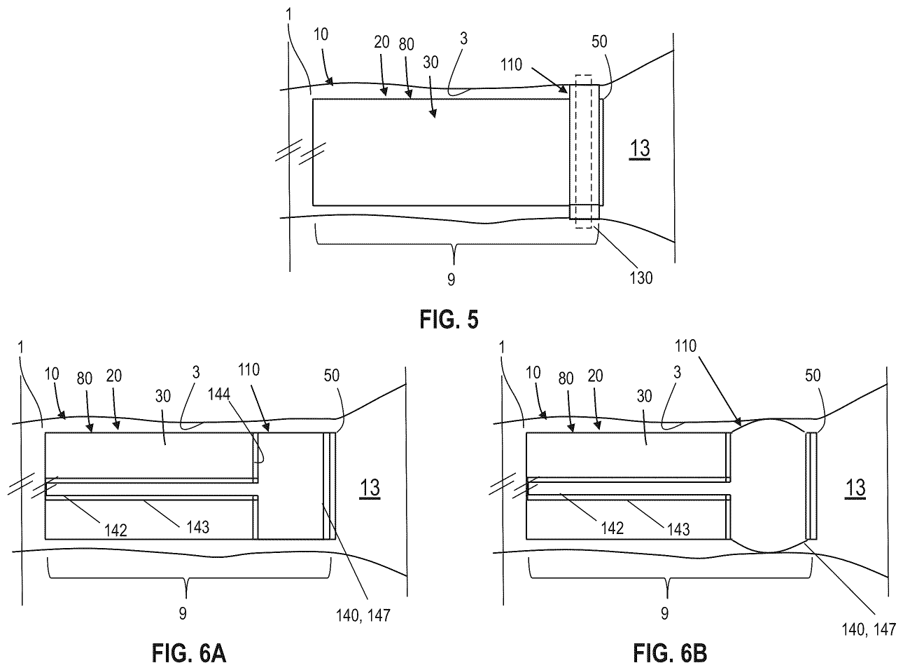

In one embodiment as shown in FIG. 3A and further in FIG. 5, the distal anchor 110 comprises a compressible ring 130 that is attached to the outer wall 80 proximate the distal end 50. The compressible ring 130 is configured for compression, including circumferential compression about a circumference of the ring against the wall 3, particularly along the isthmus 7. In this context, the extent to which the distal anchor 110 is proximate the distal end 50 is illustrated in FIGS. 3A and 4A. Note that proximate the distal end 50 may mean up to ¼, ⅓, or less than ½ the length 70 from the distal end 50. As shown in FIG. 5, as the body 30 is inserted through the eustachian tube 1 from the nasopharyngeal opening 6, it is compressed against the wall 3 within, for example, the isthmus 7, thus creating a seal between the wall 3 and the outer wall 80. In FIG. 5, the compressible ring 130 prior to compression is shown in dashed lines, and the compressible ring 130 during compression is shown in solid lines. The compressible ring 130 may be formed from any suitable flexible compressible material, such as the materials described herein for the body 30 and may include elastomers such as various open cell and closed cell foams.

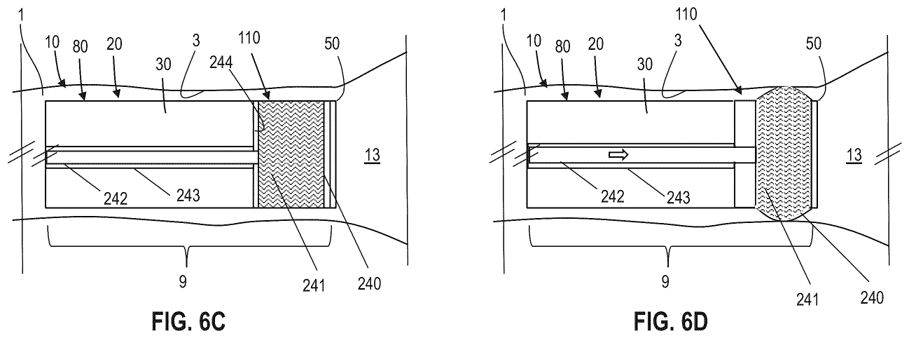

In certain embodiments, as shown in FIGS. 6A and 6B, the distal anchor 110 comprises a selectively inflatable/deflatable hollow tubular band member 140 that is attached to the outer wall 80 proximate the distal end 50. In certain embodiments, the tubular band member 140 is configured for selective inflation and deflation by a fluid, including a liquid or a gas, that flows into or is removed from the tubular band member 140, respectively, through an operable connection to a fluid conduit 142 that may in some examples be disposed within a longitudinal groove 143 in the outer wall 80 that extends to the tubular band member. The tubular band member 140 in the deflated form (FIG. 6A) may be disposed within a circumferential groove 144 so that the band member does not protrude above the outer wall 80 and interfere with the wall 3 of eustachian tube 1 during insertion of the body 30. Upon selective inflation, the tubular band member 140 expands outwardly beyond the outer wall 80 to the inflated form (FIG. 6B) and into pressed sealing engagement against the wall 3, thereby acting as the distal anchor 110.

In certain embodiments, the tubular band member 140 may be replaced by, or may house therein, a tubular or ring-shaped selectively expandable member 147, such as a sponge that is designed to expand upon absorption of a fluid, such as water or saline.

Turning to FIGS. 6C and 6D, in certain embodiments, the distal anchor 110 comprises a braided sheath member 241 that is configured for selective expansion and retraction, or contraction, by a sliding actuation member 242, including a wire or rod, that is pulled and/or pushed by a user, respectively, and may be disposed within a longitudinal groove 243 in the outer wall 80 that extends to the tubular band member 240. The braided sheath member 241 in the retracted or non-expanded form (FIG. 6C) may be disposed within a circumferential groove 244 so that the band member does not protrude above the outer wall 80 and interfere with the wall 3 during insertion of the body 30. Upon selective expansion, the braided sheath member 241 expands outwardly beyond the outer wall 80 to the expanded form (FIG. 6D) and into pressed sealing engagement against the wall 3, thereby acting as the distal anchor 110.

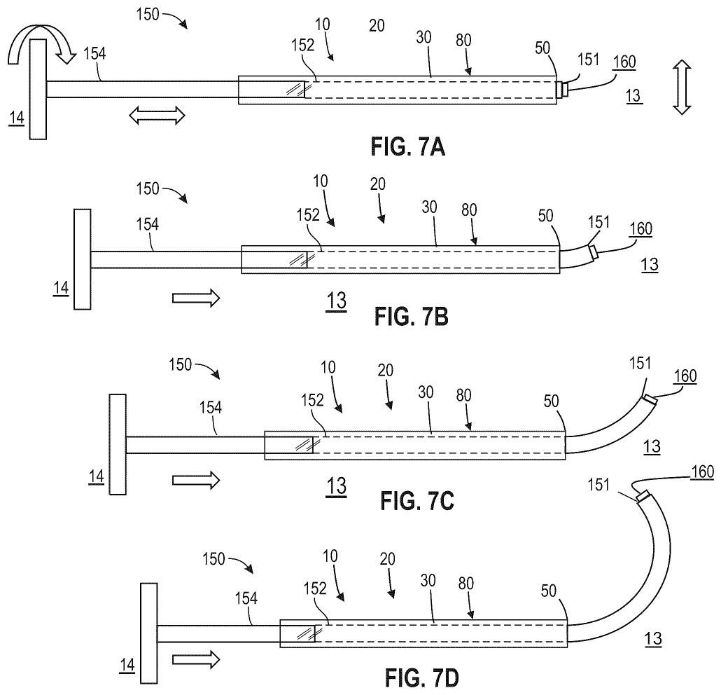

Referring now to FIGS. 7A-7D, 8A-8C, and 14, in various embodiments, the system 10 also further comprises an instrument manipulation system 150 for directional manipulation of the one or more medical devices 160 extending through the body 30, such as one or more diagnostic, treatment, and/or other surgical instruments. Such medical device(s) 160 may be configured to be movably disposed within the passageway(s) 100 of the body 30, and may comprise a single or plurality of devices in any bundle or combination, including multiples of the same device type movably disposed within one or a plurality of the passageway(s) 100 of the body 30. As used herein, movably disposed means that a distal end 151 of a medical device 160 may be longitudinally translated through, from (e.g., extended from), or into (e.g., retracted into) the passageway(s) 100, and also rotated radially clockwise or counterclockwise within the passageway(s), and angled or bent laterally (e.g., away from a central longitudinal axis of the body 30. The combination of the translation, rotation, and angulation or bending of the distal end 151 provides access to substantially all of the middle ear space 13 volume for use of the medical devices described herein.

Turning now to FIGS. 7A-7D, in certain embodiments, the instrument manipulation system 150 comprises one or more manipulators 154, wherein each manipulator 154 is operably coupled to a medical device 160 for manipulating (e.g., articulating or translating) the medical device 160. Generally, the manipulators 154 may include any suitable type of control or actuation device for manipulation of the one or more medical device(s) 160 by the user. In certain examples, the manipulator(s) 154 comprise longitudinally-extending rods, or handles, coupled to proximal end(s) 152 of the medical device(s) 160. In such embodiments, each rod 154 may be configured to be at least partially disposed within a passageway 100 in the body 30. Manual longitudinal or rotational manipulation of such rods 154 by a user may cause similar manipulation of the corresponding medical device 160. For example, distal translation of a rod 154 by the user may cause a distal end 151 of the corresponding medical device 160 to distally extend from the body 30 as shown in FIGS. 7B-7D, and proximal translation of the rod 154 may cause the distal end 151 of the medical device 160 to proximally retract back into body 30. Similarly, clockwise or counterclockwise rotation of a rod 154 may cause a corresponding rotation of the medical device 160.

In certain embodiments, as further shown in FIGS. 7B-7D, longitudinal movement of the manipulators 154 also facilitates passive lateral bending, or angular movement, of the distal end 151 of the corresponding medical device 160. In such embodiments, for example, the medical device 160 may comprise a tubular member having a preformed curvature such that proximal extension thereof from the body 30 allows the medical device 160 to curve, bend, or otherwise elastically deform into its preformed shape. In such embodiments, extent of elastic deformation of the medical device 160 is determined by the amount, degree, or radius of curvature of the preformed curvature.

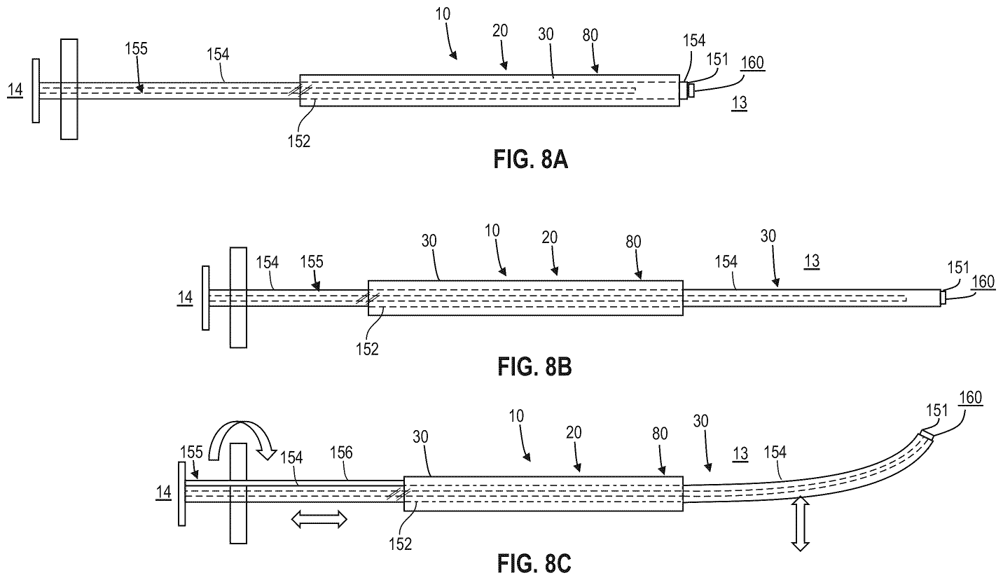

In certain embodiments, such as those of FIGS. 8A-8C, the manipulator(s) 154 may also include an active device distal end movement mechanism 155 that is configured to provide controlled lateral movement of the distal ends 151 of corresponding medical devices 160. For example, the device distal end movement mechanism 155 may include one or more separate wires or rods 156 that can be attached to a manipulator 154 proximate the device distal end 151 of a corresponding medical device 160. Upon selective, active application of a tensile force on the wire or rod, the wire or rod configured to bend the longitudinally-extending manipulator 154 and/or medical device 160 in a lateral direction to enhance access of the medical device 160 within the middle ear space by providing another degree of freedom of movement. In such embodiments, an inner or outer sheath or sleeve of the medical device 160 may include one or more lateral slots, or slits, formed therein. The one or more lateral slots may be cut through the sheath or sleeve of medical device 160 perpendicular to a bending plane, such that the slots may facilitate bending and deformation of the medical device 160 whether in response to an actuation force or external forces acting on the medical device 160.

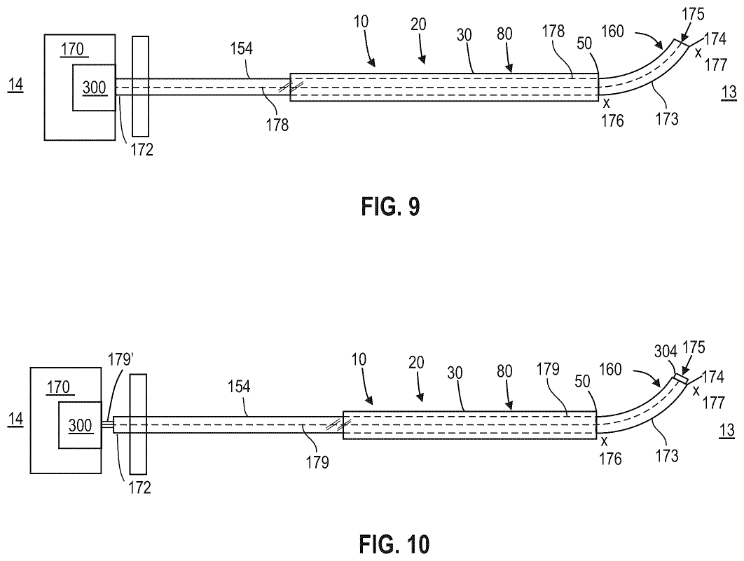

Referring now to FIGS. 9 and 10, in certain embodiments of the system 10, the medical device 160 includes an imaging device 170, such as an endoscopic or other camera, configured for attachment to the manipulator(s) 154, and having a distal end portion 174 comprising a lens 175 to facilitate imaging of the eustachian tube 1 and/or middle ear space 13. In such embodiments, the imaging device 170 may be movably disposed within a passageway 100 of the body 30 and configured for longitudinal, radial, and lateral movement of distal end portion 174 from at least a first position 176 proximate the distal end 50 of the body 30 outwardly to a second position 177 within the middle ear space 13. In certain embodiments, as shown in FIG. 9, the imaging device 170 includes an optical fiber 178, and the lens 175 comprises a distal end of the optical fiber 178 or is coupled to the distal end of the optical fiber 178, including any adaptation of the distal end to form a lens or other optical window for receiving light. In such examples, the optical fiber may be disposed within a hollow rod or sheath 173 to provide rigidity to, and to facilitate manipulation of, the optical fiber 178. The optical fiber 178 is configured for operable attachment at the proximal end portion 172 to a conventional image processing device 300.

In another embodiment, as shown in FIG. 10, the imaging device 170 comprises a signal and/or power cable 179, which may also include the hollow rod or sheath 173 that encloses signal and/or power wires 179′ of signal and/or power cable 179, and the lens 175 comprises an image sensor 304 attached to the distal end portion 174 and operably connected to the signal and/or power wires 179′. The signal and/or power cable 179 is configured for operable attachment at the proximal end portion 172 to a conventional image processing device 300. The image processing device 300 may be selected to provide imaging using any desired wavelength of electromagnetic radiation, and may include infrared, visible, and ultraviolet radiation.

As described above, the embodiments of FIGS. 9 and 10 provide an imaging device 170 feature that enables visualization and examination of the middle ear space 13. In certain embodiments, the system 10 further provides illumination that may be needed for visualization of the middle ear space 13, as described in more detail below.

Generally, the maneuverable imaging device 170 described herein enables still camera and/or video visualization in all directions, thereby providing a surgeon with a good view into the middle ear structures found in the middle ear space 13. Such visualization of the eustachian tube 1 and middle ear space 13, in turn, facilitates all manner of benefits related to assessment, diagnosis, treatment and surgical procedure within both the eustachian tube 1 and the middle ear space 13. For example, the adjustability of the imaging device 170 provides intra-operative camera visualization for the provision of different angles and viewpoints, thereby providing additional visual information which may be critical for deciding the next surgical step or for deciding whether the treatment is carried out correctly (e.g., no more cholesteatoma in middle ear space, correct prosthesis placement and function, graft placement, making sure there are no other unexpected conditions, etc.). In some examples, integration of imaging device 170 into system 10 facilitates pre-operative, intra-operative, and/or post-operative camera visualization (e.g., video capture), for documentation and demonstration of ossicle function (or other diagnostic information) before, during, and after performance of a surgical procedure. For example, pre-operative camera visualization may add vital information to better determine the patient's condition and whether surgery is needed or not (e.g., small cholesteatomas that are not detectable by CT (Computed Tomography) scans may be detected using the non-surgical camera visualization through the eustachian tube 1, etc.). Additionally, post-operative camera visualization may show how well a previous surgery was carried out, and whether a follow-up surgery is required or not (e.g., ossicular chain reconstruction verification, re-occurrence of a cholesteatoma, etc.).

In further examples, the integration of the imaging device 170 into system 10 provides non-surgical, visual access to the eustachian tube 1 and middle ear space 13, thereby enabling better decision-making based on real-time information before surgical intervention, such as, for example, the selection of an appropriate surgical approach from the start instead of switching the surgical approach during surgery due to new information obtained about the middle ear space 13 during the surgery, or instead of selecting a more invasive approach than necessary (e.g., selecting postauricular, even though transcanal surgery would have been sufficient).

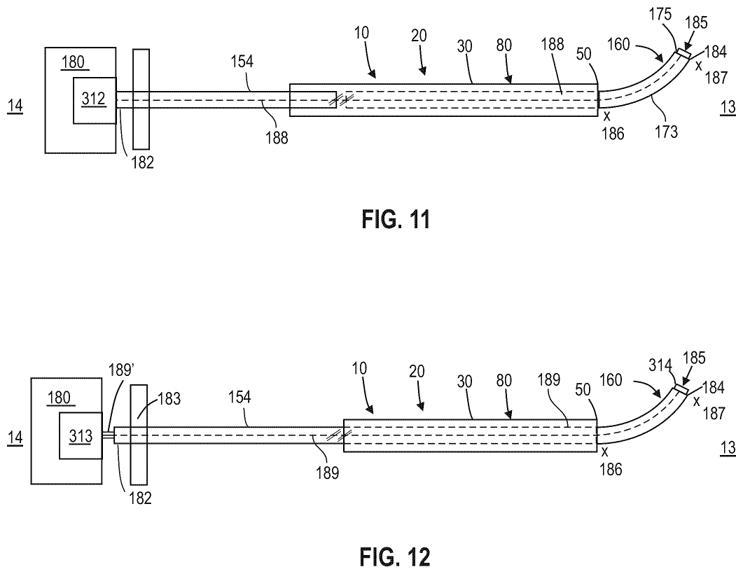

Referring now to FIGS. 11 and 12, in certain embodiments of the system 10, the medical device 160 includes an illuminator 180 configured for attachment to the manipulator(s) 154, and having a distal end portion 184 comprising a light emitting portion 185, for purposes of illuminating the eustachian tube 1 and/or middle ear space 13. In such embodiments, the illuminator 180 is movably disposed within a passageway 100 and configured for longitudinal, radial, and lateral movement of the light emitting portion 185 from a first position 186 proximate the distal end 50 of the body 30 outwardly to a second position 187 within the middle ear space 13.

In certain embodiments, as shown in FIG. 11, the illuminator 180 comprises an optical light fiber 188, and the light emitting portion 185 comprises an exposed distal end of the optical light fiber 188. In certain embodiments, the distal end of the optical light fiber 188 is disposed adjacent to the lens 175, which may be coupled to the distal end of the optical light fiber 188, including any adaptation of the distal end of the optical light fiber 188 to form a lens or other optical window for transmitting light. In such examples, the optical light fiber 188 may be disposed within the hollow rod or sheath 173 to provide rigidity to, and to facilitate manipulation of, the optical light fiber 188.

The optical light fiber 188 is configured for operable attachment at the proximal end portion 182 to a light source 312, such as an LED (light emitting diode) light source that is configured to provide various wavelengths of light for illumination of the middle ear space 13, or a laser light source that is configured for various laser-based surgical procedures on the tissues disposed within the middle ear space 13, such as a surgical laser for ablation or excision of various tissues, including bone, cartilage, and other tissues. The optical light fiber 188 may comprise a single optical fiber or a plurality of fibers, such as a bundle of fibers, which may be controlled and operated as a unit, or in one or more groups or clusters, or individually, with regard to the wavelength and intensity of the light emitted.

In certain embodiments, as shown in FIG. 12, the illuminator 180 comprises a signal and/or power cable 189, which may also comprise the hollow rod or sheath 173 that encloses signal and/or power wires 189′ of the cable 189, and the light emitting portion 185 comprises a light emitting device 314, such as an LED, attached to the distal end portion 184 and operably connected to the signal and/or power cable 189. The light emitting device 314 may comprise a single device or a plurality of devices, such as an array of devices, which may be controlled and operated as a unit, or in one or more groups or clusters, or individually, with regard to the wavelength and intensity of the light emitted. The signal and/or power cable 189 may be operably attached at the proximal end portion 182 to a power source device 313, or to a power and/or signal control device that is configured to operate the light emitting device 314.

The embodiments of the system 10 depicted in FIGS. 11 and 12 can be extended from the body 30 through a passageway 100 and into the middle ear space 13 to illuminate the middle ear space 13. With the directional adjustability of illuminator 180 described herein, most of the middle ear space 13 can be illuminated during a surgical procedure. Such illumination provides several advantages and benefits, including: illumination for imaging/camera features incorporated in the system 10, improved illumination, including more consistent illumination with regard to intensity and illumination density and more elimination of shadows within of middle ear space 13 than is achievable by conventional devices, such as microscope illumination through the narrow ear canal, wherein instruments inserted through ear canal are known to block part of the illumination from the microscope resulting in shadows in the middle ear space 13 obstructing the visualization for surgical manipulation and procedures.

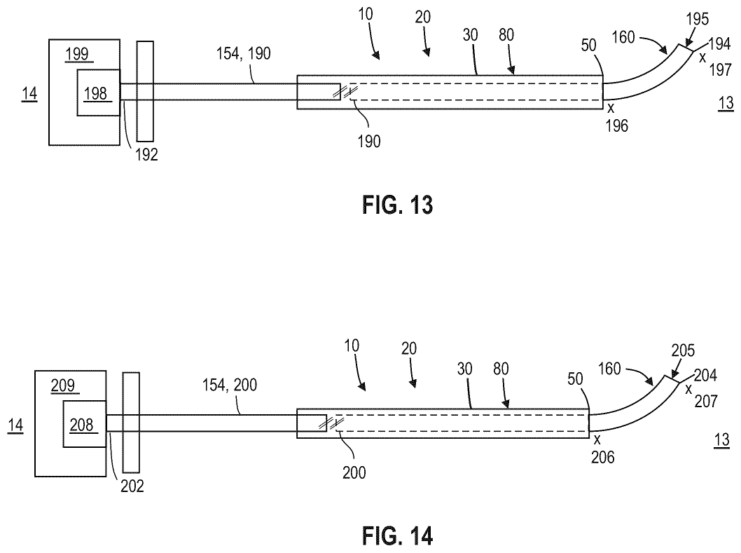

Referring to FIG. 13, in certain embodiments of the system 10, the medical device 160 comprises a suction tube 190 configured for attachment to manipulator(s) 154, and having a distal end portion 194 comprising a distal opening 195. The suction tube 190 may be movably disposed within a passageway 100 and configured for longitudinal, radial, and lateral movement of the distal opening 195 from a first position 196 proximate the distal end 50 of the body 30 outwardly to a second position 197 within the middle ear space 13. In certain embodiments, the suction tube 190 is configured for attachment on the proximal end portion 192 to a vacuum source 198, which together with the suction tube 190 forms a trans-eustachian suction system 199. The distal end portion 194 of the suction tube 190 is configured for removal of a fluid or tissue, or a combination thereof, from the middle ear space 13 through the distal opening 195. The vacuum source 198 may include any suitable type of vacuum or suction device, such as a suction pump or aspirator.

In some examples, the suction tube 190 may be used, for example, to dry out the middle ear space 13 in cases of chronic otitis without the need of a surgery (e.g., myringotomy, etc.). Additionally, the suction tube 190 can be used during surgeries to dry out the middle ear space 13 for better visibility and better surgical manipulation.

Referring to FIG. 14, in certain embodiments of the system 10, the medical device 160 comprises an irrigation tube 200 having a proximal end portion 202 configured for attachment to manipulator(s) 154, and having a distal end portion 204 comprising a distal opening 205. The irrigation tube 200 may be movably disposed within a passageway 100 and configured for longitudinal, radial, and lateral movement of the distal opening 195 from a first position 206 proximate the distal end 50 of the body 30 outwardly to a second position 207 within the middle ear space 13. In certain embodiments, the irrigation tube 200 is configured for attachment on the proximal end portion 202 to a fluid source 208 and forms a trans-eustachian irrigation system 209, and the distal end portion 204 is configured for infusion of a fluid, such as an irrigation fluid or a medicament fluid, into the middle ear space 13 through the distal opening 205.

In some examples, the irrigation tube 200 can be used for delivery of irrigation fluids or therapeutic medicaments to the middle ear space 13. Additionally, the irrigation tube 200 can be used to deliver stains and dyes that improve visualization or facilitate marking and identification of structures in the middle ear space 13 (e.g., via autofluorescence, coloring of certain tissue, etc.) for CT, MRI (magnetic resonance imaging), other types of imaging, and/or during surgery.

In certain embodiments of the system 10, the suction or irrigation tube (190 or 200) comprises a plurality of suction and/or irrigation tubes in combination. In such embodiments, a proximal end of at least one tube is configured for fluidic coupling to the fluid source 208, and/or a proximal end of at least one tube is configured for fluidic coupling to the vacuum source 198, and the respective distal ends are configured for infusion of fluids, removal of a fluid or tissue, or a combination thereof, from the middle ear space 13. A combination of the suction tube 190 and the irrigation tube 200 in the system 10 makes it is possible to deliver fluids to rinse the middle ear space 13 during surgery (e.g., delivery of BSS (balanced salt solution), saline, PFO (Perfluoro-N-octane), air, etc.) via the irrigation tube 200, while also suctioning such delivered fluids, in addition to other materials in the middle ear space 13, through the suction tube 190. This enables a stable surgical working environment and all undesired materials (e.g., blood, excised tissue, etc.) can be continuously removed.

In other examples, when the suction tube 190 or the irrigation tube 200 is used in combination with an additional suction or irrigation device inserted at the ear canal, it is possible to rinse the middle ear space 13 during surgery (e.g., using BSS, saline, PFO, air, etc.) from the eustachian tube 1 via the irrigation tube 200, and continuously suction out such fluids and other materials (e.g., blood, tissue, etc.) through the ear canal with the additional device, or vice-versa. This similarly enables a stable surgical working environment and all undesired materials (e.g., blood, tissue, etc.) being continuously removed.

The integration of the suction and/or infusion tube (190 or 200) with system 10 provides the following benefits and advantages: 1) enabling the delivery of therapeutic substances through infusion, whether during a surgery or a treatment procedure; 2) facilitating improved diagnostic visualization (e.g., CT, MRI, etc.) by infusing stains and dyes into the middle ear space 13 (e.g. autofluorescence, coloring of certain tissues, etc.); 3) enabling infusion into or aspiration of fluids from (e.g., BSS, saline, PFO, air, etc.) the middle ear space 13 during surgery to provide a stable surgical working environment and for continuous removal of undesired substances (e.g., blood, tissue, debris, etc.); 4) enabling aspiration to actively dry out the middle ear space without surgery (e.g., myringotomy, tympanoplasty, etc.) in cases of chronic otitis; 5) enabling aspiration during surgery to dry out the middle ear space for better visibility and better surgical manipulation; 6) eliminating the need to perforate the eardrum for access to the middle ear space, such as by use of a needle to provide medicaments or contrast stains and dyes, with the concomitant elimination of risk of trauma to the middle ear structures, such as the ossicle chain during such procedures.

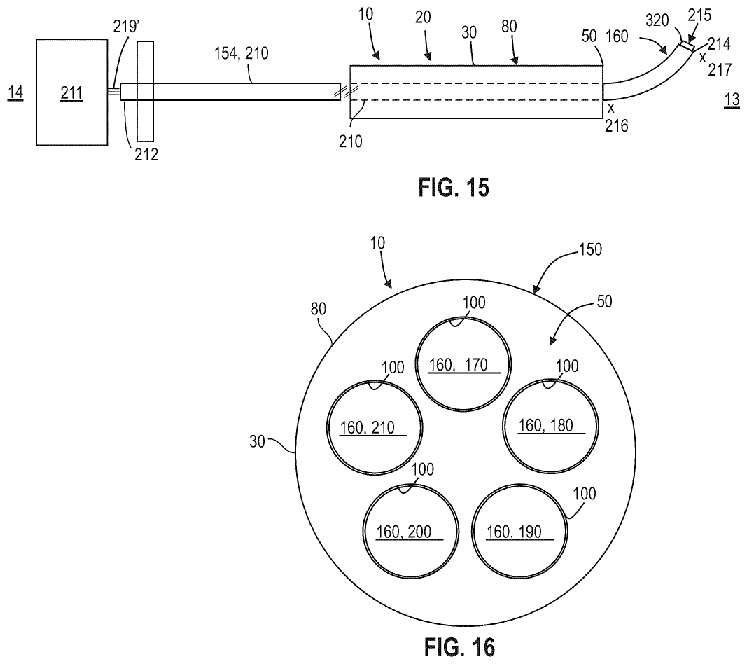

Referring to FIG. 15, in certain embodiments of the system 10, the medical device 160 comprises an audio transmitter tube 210 having a proximal end portion 212 configured for attachment to the manipulator(s) 154, and having a distal end portion 214 comprising an audio-emitting portion 215. The audio transmitter tube 210 may be movably disposed within a passageway 100 and configured for longitudinal, radial, and lateral movement of the light emitting portion from a first position 216 proximate the distal end 50 of the body 30 outwardly to a second position 217 within the middle ear space 13. In certain embodiments, as shown in FIG. 15, the audio transmitter tube 210 includes a signal and/or power cable 219, which may also include a hollow rod or sheath that encloses signal and/or power wires 219′ of the cable 219, and the audio-emitting portion 215 includes an audio-emitting (or audio-transmitting) device 320, such as a miniature speaker or radio to audio frequency transducer, attached to the distal end portion 214 and operably connected to the signal and/or power cable 218. The audio-emitting device 320 may comprise a single device or a plurality of devices, such as an array of devices, which may be controlled and operated as a unit, or in one or more groups or clusters, or individually, with regard to the wavelength, frequency, and intensity of the audio emitted. The signal and/or power cable 218 may be operably attached at the proximal end portion 212 to a power source device and/or audio source 211, or to a power and/or signal control device that is configured to operate the audio-emitting device 320.

The audio transmitter tube 210 can be extended from the body 30 through a passageway 100 into the middle ear space 13 to stimulate structures within the middle ear space 13 with audio signals. The stimulation of the middle ear structures, coordinated with the illumination, visualization, and other features described herein, enables direct observation of the response of the middle ear structures to a wide spectrum of audio signals for diagnostic, assessment, and treatment purposes, as well as during a surgery when a patient may be anesthetized and unable to confirm receipt of an auditory input. The localized audio input may also enable such input to be performed in an integrated manner without the need for other testing and assessment equipment and/or facilities. In some examples, in order to ensure that the response of the middle ear structures to audio stimulation is due to the audio input received from the audio transmitter tube 210 disposed through the eustachian tube 1, the audio transmitter tube 210, and thus, system 10, may be used in combination with easily accessible and inexpensive audio blocking/dampening mechanisms for blocking noise the though the ear canal (e.g., audio isolating or blocking headphones).

Referring to FIG. 16, in certain embodiments of the system 10, the body 30 comprises the elements of some or all of the subsystems of the system 10 disclosed herein, and comprises a multi-function medical system that enables all manner of medical assessment, diagnostic, treatment, and/or surgical procedures in the eustachian tube 1 and/or middle ear space 13, including the middle ear structures disposed therein, by incorporation of an imaging device 170, an illuminator 180, a suction tube 190, an irrigation tube 200, and/or an audio transmitter tube 210 into a single medical system. FIG. 16 also demonstrates that in certain embodiments, any single medical device 160, or any combination of these medical devices 160, may be incorporated together into a single medical system, such as might be desirable to accomplish specific medical/surgical procedures, and that these embodiments may include more than one of any particular device (e.g., two imaging devices, three illuminators, etc.).

Referring to FIGS. 17A-18C, in certain embodiments of the system 10, an instrument housing 400 disposed proximal to the body 30 and/or tubular member 32 is configured to sheathe, or contain, one or more of the medical devices 160 described herein before such medical devices 160 are movably deployed into the body 30 during a surgical procedure. In such embodiments, the instrument housing 400 enables a multi-function medical system that enables all manner of medical assessment, diagnostic, treatment, and/or surgical procedures in the eustachian tube 1 and/or middle ear space 13, including the middle ear structures disposed therein, by incorporation of an imaging device 170, an illuminator 180, a suction tube 190, an infusion tube 200, and/or an audio transmitter tube 210 into a single medical system.

In these embodiments, a particular combination of medical devices 160 may be deployed from the instrument housing 400 and into the body 30 one at a time from a single source and in any sequence. In the embodiments of FIGS. 17A-18C, the medical devices 160 are housed in an instrument housing 400 that is disposed ex vivo in the external environment 14 during a surgical procedure for use by a surgeon or other medical professional and is in operative engagement with and attachment to tubular member 32 and/or body 30 that extends into the eustachian tube 1 (e.g., FIGS. 3A and 4A). The instrument housing 400 is configured to receive and house a plurality of medical devices 160, which may include any suitable number of medical devices 160. Any suitable medical device 160 that is capable of being deployed from the instrument housing 400 and through the body 30 in the manner described herein may be used therewith, including those specifically listed above. In certain embodiments, the number of instruments 160 may range from 2-20, and more specifically 2-10, and even more specifically 2-5. The instrument housing 400 may have any suitable size, shape, or instrument deployment mechanism, including those of FIGS. 17A-18C. The instrument housing 400 disclosed herein may be used with a body 30 that comprises a single passageway 100, including the embodiments illustrated in FIGS. 3B and 4B as described herein.

Referring now to FIGS. 17A-17C, in certain embodiments, the instrument housing 400 may comprise a fixed housing 402, which may have one or more tapered portions in some examples. Fixed housing 402 comprises a first portion 406, in the embodiment illustrated as a cylindrical portion, that extends from a housing proximal end 410 to a second portion 412, in the embodiment illustrated a tapered and frusto-conical portion, that extends to the housing distal end 416. The first portion 406 and the second portion 412 may be formed as a unitary piece, such as by 3D printing, or as separate pieces that may be attached or joined together by any suitable joining or attachment method or mechanism. The instrument housing 400 comprises a plurality of instrument chambers 418 disposed in the first portion 406 that are configured to receive a corresponding plurality of medical devices 160 at a housing proximal end 410, including in certain embodiments an imaging device 170, an illuminator 180, a suction tube 190, an irrigation tube 200, and/or an audio transmitter tube 210. In FIGS. 17A and 17B, the instrument chambers 418 are shown in the form of cylindrical bores extending through first portion 406. The instrument chambers 418 may comprise any predetermined number and have any predetermined size or cross-sectional shape, and may be disposed within the instrument housing 400 in any predetermined pattern, including a radially equally spaced array of five (5) instrument chambers 418 as shown in FIG. 17B. The instrument chambers 418 may have the same size and shape, or different sizes and shapes, so long as the sizes and shapes permit translation and deployment of the medical device 160 into and through the tubular member 32 and body 30 into the eustachian tube 1 or middle ear space 13 as described herein.

The second portion 412 comprises a tapered bore 422, such as a frustoconical-shaped bore, defined by a first boring opening 426 and that tapers inwardly toward longitudinal axis 428 along bore sidewall 430 to a second bore opening 432. The instrument housing 400 is attached to tubular member 32 with the second bore opening 432 opening into the inside diameter 434 of the tubular member 32. The instrument housing 400 may be formed from any suitable biocompatible material, including biocompatible metals, such various grades of stainless steel, nitinol, or titanium alloys, or engineering thermoset or thermoplastic polymers.

The fixed housing 402 may be used by sequentially translating one of the medical devices 160 that has been disposed within an instrument chamber 418 forward and toward the housing distal end 416. As the medical device 160 translates forward, in many instances it will engage the bore sidewall 430 and be deflected inwardly toward the longitudinal axis 428, through second bore opening 432, and into tubular member 32 as shown in FIG. 17C. As forward translation continues, the medical device 160 passes through a singular passageway 100 of the body 30 for use in a medical procedure within the eustachian tube 1 and/or the middle ear space 13, according to the intended purpose of the medical device 160. Once the procedure or function is completed, the medical device 160 is translated rearward and withdrawn back into the respective instrument chamber 418. The other medical devices 160 may then be operated in the same manner to access the eustachian tube 1 and/or middle ear space 13 in any predetermined sequence of use to complete one or more medical procedures.

Referring now to FIGS. 18A-18C, in certain embodiments, the instrument housing 400 may comprise a partially rotatable and partially fixed housing 442, which may be cylindrical in shape in some examples. Housing 442 comprises a rotatable first portion 446, in the embodiment illustrated a rotatable cylindrical portion, that extends from a housing proximal end 450 to a fixed second portion 452, in the embodiment illustrated a fixed cylindrical portion, that extends to the housing distal end 456. The rotatable first portion 446 and the fixed second portion 452 may be formed as separate pieces, such as by 3D printing, that are attached by journal pin 457 that is fixed to the fixed second portion 452 and which is rotatably attached to the rotatable first portion, thereby allowing the rotatable first portion 446 to rotate around the journal pin 457. Depending on the material used to make first portion 446 to rotate around the journal pin 457, a rotation device such as a bushing(s) or bearing(s) may be disposed between them to facilitate rotation. For example, a plastic first portion 446 may be able to rotate about a metal pin with no rotation device, while a metal first portion 446 may benefit from disposing a rotation device or devices between them. Journal pin 457 may also include various features on opposing ends thereof, such as a pin head that is wider than the remaining journal pin diameter in order to retain the rotatable first portion 446 and fixed second portion 452.

The instrument housing 400 comprises a plurality of instrument chambers 458 disposed in the rotatable first portion 446 and that extend through the rotatable first portion 446 and are configured to receive the insertion of a corresponding plurality of medical devices 160 at a housing proximal end 450, such as those shown in FIGS. 18A and 18B, including in certain embodiments an imaging device 170, an illuminator 180, a suction tube 190, an irrigation tube 200, and/or an audio transmitter tube 210. In FIGS. 18A and 18B, the instrument chambers 458 are shown in the form of cylindrical bores extending through rotatable first portion 446. The instrument chambers 458 may comprise any predetermined number and have any predetermined size or cross-sectional shape. Further, the instrument chambers 458 may be disposed within the instrument housing 400 in any predetermined pattern, including a radially equally spaced array of five (5) instrument chambers 458 as shown in FIG. 18B. The instrument chambers 458 may have the same size and shape, or different sizes and shapes, so long as the sizes and shapes permit translation and deployment of the medical device 160 into and through the tubular member 32 and body 30 into the eustachian tube 1 or middle ear space 13, as described herein.

The fixed second portion 452, including fixed portion 454, comprises a mating bore 422, such as a cylindrical mating bore, defined by a first bore opening 466 and that extends parallel to and radially offset from longitudinal axis 468 along bore sidewall 470 to a second bore opening 472. The instrument housing 400 is attached to tubular member 32 with the second bore opening 472 opening into the inside diameter 434 of the tubular member 32. The instrument housing 400 in such embodiments may also be formed from any suitable biocompatible material, including biocompatible metals, such various grades of stainless steel, nitinol, or titanium alloys, or engineering thermoset or thermoplastic polymers.

The rotatable first portion 446 may be rotated manually by a surgeon or may be attached to and rotated automatically by a computer-controlled drive motor. The medical device 160 may be inserted, retracted, and rotated manually by a surgeon or may be inserted, retracted, and rotated automatically by a computer controlled robotic system. In certain embodiments, the outer surface 474 of the rotatable first portion 446 includes gripping features 476 to enhance grasping and manipulation by a human hand and fingers, such as various forms or patterns of knurling or radially spaced, longitudinally-extending grooves.

The partially rotatable and partially fixed housing 442 may be used by sequentially translating one of the medical devices 160 that has been disposed within an instrument chamber 418 forward toward the housing distal end 456. As the medical device 160 translates forward, it passes into and through second bore opening 472 and tubular member 32. As forward translation continues, the medical device 160 passes through the singular passageway 100 of the body 30 for use in a medical procedure within the eustachian tube 1 and/or the middle ear space 13 according to the intended purpose of the medical device 160. Once the procedure is completed, the medical device 160 is translated rearward and withdrawn back into the respective instrument chamber 458. The other medical devices 160 may then be operated in the same manner to access the eustachian tube 1 and/or middle ear space 13 in any predetermined sequence of use to complete one or more medical procedures.

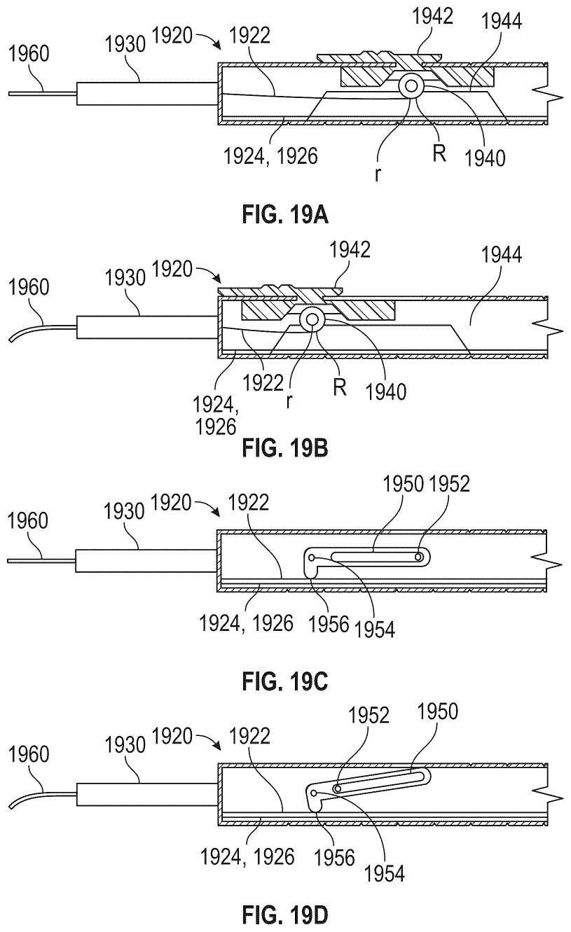

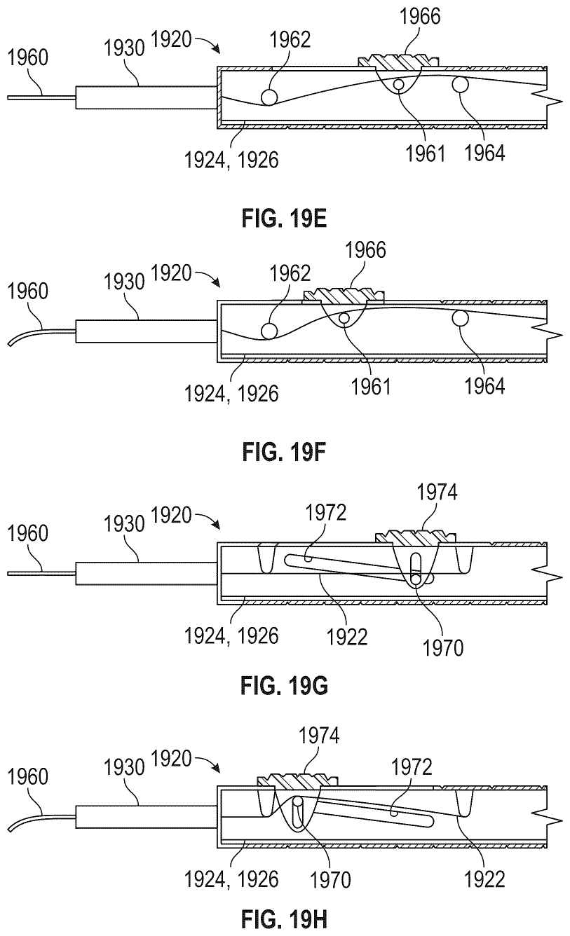

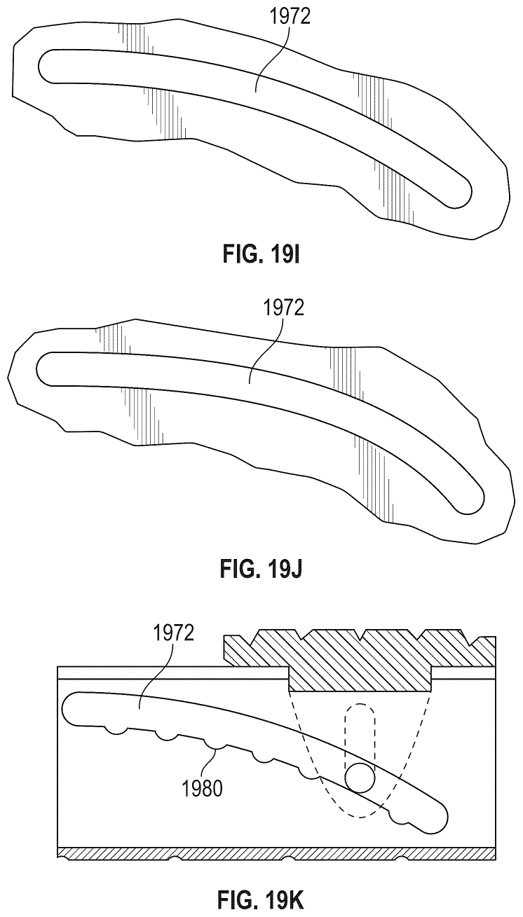

Referring now to FIGS. 19A-19K, in accordance with certain embodiments of a trans-eustachian probe (hereinafter, “probe”) 1920 utilizing a pull-wire 1922 for actuation of, e.g., medical device 1960, various mechanisms, as will be discussed in further detail below, can be used for increasing the tension of the pull-wire 1922. By way of example, as illustrated in FIGS. 19A-19K, the probe 1920 can include a trans-eustachian body 1930 having two optical fibers 1924 and 1926 extending therethrough (e.g., for imaging and/or illumination the middle ear space). In some embodiments, manipulators 154 for manipulating medical devices 160, described above, can take advantage of the mechanisms described below with respect to the pull-wire 1922.

In FIGS. 19A and 19B, the pull-wire 1922 is wound on a pinion 1940 secured between a control button 1942 and a base 1944. The pinion 1940 includes two surfaces, a smaller diameter surface r which rolls between the control button 1942 and a base 1944, and a larger diameter surface R about which the pull wire 1922 winds. The radial difference between the smaller and larger diameter surfaces r and R results in a differential displacement Δ1 in the pull wire as the pinion 1940 rotates and translates. By selecting appropriate diameters for the smaller and larger diameter surfaces r and R, a relatively small amount of pull wire displacement Δ1 can be achieved during a relatively large amount of control button translation, providing the user with precise control over the deflection in medical device 1960, which may include a slotted tip design. In one embodiment, the smaller diameter surface r includes gear teeth with mating gear teeth on the control button 1942 and the base 1944. This may reduce the likelihood of slippage.

FIGS. 19C and 19D illustrate a lever arm 1950 with a sliding actuation pin 1952 held in place by a fixed pin 1954 at a pivot of the arm. A control button can be used to advance the sliding pin 1952, permitting the proximal portion of the lever arm 1950 to rise, thus rotating a lanyard 1956 at a distal end of the lever arm 19 to apply tension to the pull-wire 1922. FIGS. 19E and 19F show a pull-wire 1922 threaded over a sliding pin 1961 and a first fixed pin 1962 and anchored to a second fixed pin 1964. Advancing a control button 1966 attached to the sliding pin 1961 increases the tension in the pull-wire 1922.

FIGS. 19G and 19H illustrate a pull-wire 1922 threaded over a sliding pin 1970 that is directed in a generally upward direction by a guide track 1972 as a control button 974 is advanced. The path of the guide track 1972 determines how the tension in the pull-wire 1922 varies as the control button is advanced, thus providing a smooth and controlled increase in tension. In the case of a linear guide, like the one illustrated in FIGS. 19G and 19H, the pull-wire take up will occur in the latter portion of the advancement of the control button 1974. In the alternative configuration shown in FIG. 191, the guide track 1972 is reshaped to provide greater take-up of the pull-wire at the beginning of the advancement by the control button 1974 to produce a more balanced increase in tension throughout the stroke of the control button 1974. In FIG. 19J, the guide track 1972 inclines even more sharply so that most of the tension increase takes place early in the stroke of the control button 1974. FIG. 19K illustrates an alternative embodiment of the guide track 1972 with detents 1980, allowing for distinct “stops” along the path corresponding to different angles of the medical device 1960. A shelf or surface with detents can be also be used with any of the embodiments of the otologic instruments presented herein using a sliding pin or similar actuation mechanism, including any of the embodiments shown in FIGS. 19A-19K.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A trans-eustachian multi-function system, comprising:
a body configured for disposition within a eustachian tube, the body comprising:
a proximal end,
a distal end,
a transverse cross-sectional shape shaped to correspond with a cross-section of the eustachian tube,
a longitudinally-extending length,
an outer wall configured for disposition proximate a wall of the eustachian tube, and
an inner portion defining a passageway; and
a distal anchor disposed proximate the distal end, the distal anchor configured to releasably anchor the body against the wall of the eustachian tube.

2. The trans-eustachian multi-function system of claim 1, further comprising an imaging device having a proximal end portion configured for attachment to a manipulation device and a distal end portion comprising a lens, the imaging device movably disposed within the passageway configured for longitudinal, radial, and/or lateral movement of the lens from a first position proximate the distal end of the body outwardly to a second position within a middle ear space.

3. The trans-eustachian multi-function system of claim 2, wherein the imaging device comprises an endoscopic camera.

4. The trans-eustachian multi-function system of claim 1, further comprising an illuminator having a proximal end portion configured for attachment to a manipulation device and a distal end portion comprising a light emitting portion, the illuminator movably disposed within the passageway configured for longitudinal, radial, and/or lateral movement of the light emitting portion from a first position proximate the distal end of the body outwardly to a second position within a middle ear space.

5. The trans-eustachian multi-function system of claim 4 wherein the illuminator comprises an optical light fiber.

6. The trans-eustachian multi-function system of claim 4 wherein the illuminator comprises a light emitting device disposed at the light emitting portion.

7. The trans-eustachian multi-function system of claim 1, further comprising an audio-transmitting device having a proximal end portion configured for attachment to a manipulation device and a distal end portion comprising an audio-emitting portion, the audio-transmitting device movably disposed within the passageway configured for longitudinal, radial, and/or lateral movement of the audio-emitting portion from a first position proximate the distal end of the body outwardly to a second position within a middle ear space.

8. The trans-eustachian multi-function system of claim 1, further comprising a suction tube or infusion tube having a proximal end portion configured for attachment to a manipulation device and a distal end portion comprising a distal opening, the suction tube or infusion tube movably disposed within the passageway configured for longitudinal, radial, and/or lateral movement of the distal opening from a first position proximate the distal end of the body outwardly to a second position within a middle ear space.

9. The trans-eustachian multi-function system of claim 8 wherein the suction tube or infusion tube comprises the suction tube, and wherein the suction tube is configured for attachment of the proximal end portion to an evacuation device, and the distal end is configured for removal of a fluid or tissue, or a combination thereof, from the middle ear space.

10. The trans-eustachian multi-function system of claim 8 wherein the suction tube or infusion tube comprises the infusion tube, and wherein the infusion tube is configured for attachment of the proximal end portion to a fluid infusion device, and the distal end is configured for infusion of a fluid into the middle ear space.

11. The trans-eustachian multi-function system of claim 1, further comprising a tubular member attached to the proximal end of the body, the tubular member extending to and attached to an instrument housing configured to house a plurality of medical instruments, the plurality of medical instruments configured for selective insertion through an inner diameter of the tubular member and the passageway into a eustachian tube and/or a middle ear space.

12. The trans-eustachian multi-function system of claim 1, wherein the distal anchor comprises a selectively expandable seal member or a compressible seal member.
